(19) United States
(12) Patent Application Publication
(10) Pub. No.: US 2014/0045788 A1
(43) Pub. Date: Feb. 13, 2014

(54) METHOD OF STABLY TREATING INCONTINENCE USING A BULKING AGENT

(71) Applicant: Howard Franklin, Richboro, PA (US)

(72) Inventor: Howard Franklin, Richboro, PA (US)

(73) Assignee: SALIX PHARMACEUTICALS, LTD., Raleigh, NC (US)

(21) Appl. No.: 14/058,836

(22) Filed: Oct. 21, 2013

Related U.S. Application Data
(63) Continuation of application No. 13/788,401, filed on Mar. 7, 2013.
(60) Provisional application No. 61/665,846, filed on Jun. 28, 2012.
(60) Provisional application No. 61/731,774, filed on Nov. 30, 2012.

(51) Int. Cl. A61K 31/721

(52) U.S. Cl. CPC A61K 31/721
USPC 514/59

(57) ABSTRACT

The invention provides a method for treating fecal incontinence in a subject comprising administering in an appropriate subject a therapeutically effective amount of a biocompatible bulking agent to the wall of the anal canal. The biocompatible bulking agent comprises dextranomer and a pseudoplastic carrier, such as, hyaluronic acid, and is stable and effective in treating fecal incontinence for over 24 months, and up to at least 36 months or more following an initial administration.

### RELATED APPLICATIONS

This application is a continuation of U.S. application Ser. No. 13/788,401, filed Mar. 7, 2013, which claims the benefit of U.S. Provisional Application No. 61/665, 846, filed Jun. 28, 2012 and U.S. Provisional Application No. 61/731,774, filed Nov. 30, 2012. The entire contents of each of the aforementioned applications are incorporated herein by reference.

### BACKGROUND

Incontinence refers to the loss of bowel control and can be caused by numerous underlying conditions, including, for example, injury, childbirth in women, neurological disease, infection, and degenerative changes associated with aging.

Fecal incontinence is the inability to control bowel movements causing the unwanted and uncontrollable release of stool from the rectum passing through the anus. Fecal incontinence can include, for example, solid, liquid and gas excretions, or mixtures thereof, during loss of sphincter control. The severity of incontinence varies from an occasional leakage of stool in combination with passing gas to complete loss of bowel control such that feces may escape the rectum and anus in an uncontrolled manner. Normal bowel control requires a number of properly working functionalities, including a properly functioning small intestine, large intestine, rectum, anal sphincter muscles, and the nervous system. The amount of stool and its consistency also affects a person's ability to control bowel movements. Damage to or disease involving any of these systems may result in fecal incontinence. Fecal incontinence may be caused by a variety of underlying conditions, which may include chronic constipation, diarrhea, muscle or nerve damage, weakened anal sphincter muscle associated with injury or aging, colorectal cancer, and in women, damage from giving birth. Other causes may include anal surgery, prostrate surgery in men, spinal cord injury, stroke, a systemic disease, such as multiple sclerosis, and Parkinson's disease, rectal prolapse, overuse of laxatives, old age, major depression, Irritable Bowel Syndrome, and Crohn's Disease.

Treatment for fecal incontinence varies widely, beginning with adjustments made to behaviors and diet. For example, various lifestyle modifications, such as diet, fluid intake, regular exercise, and regular bowel habits, can be used as a first resort to treating fecal incontinence. Other options for include surgical and/or pharmaceutical treatments, both of which may be more severe and which may be associated with unwanted side effects and toxicities.

Non-invasive solutions that are stable, safe, and effective in treating fecal incontinence are highly desired, and in particular, solutions which provide a simple and fast administration scheme and which provides for lasting benefit for at least 12, more preferably at least 24 months, or even more preferably at least 36 months after treatment with out the need for reintervention.

### SUMMARY

Embodiments relate to the use of biocompatible compositions for treating incontinence, and more particularly, for treating fecal incontinence. Fecal incontinence can include, for example, solid, liquid and gas excretions, or mixtures thereof, during loss of sphincter control. More particularly, embodiments relate to the safe and effective use of a biocompatible bulking agent for the treating of fecal incontinence for a period up to at least 36 months without further treatment, including incontinence reintervention.

Disclosed herein are methods for safely and effectively treating incontinence, e.g., fecal incontinence, in a non-invasive manner (i.e., without requiring surgery) in a subject with incontinence involving the administration of a biocompatible bulking agent composition directly to the affected target tissue. For example, with fecal incontinence, the biocompatible bulking agent can be administered, e.g., by injection or catheter, to the wall of the anal opening, e.g., to the anal sphincter region. The biocompatible bulking agent can comprise a dextranomer and a pseudoplastic carrier, such as, hyaluronic acid, and is preferably stable and effective in treating fecal incontinence for over 24 months, or up to at least 36 months or more following an initial administration.

In one aspect, provided herein is a method for treating fecal incontinence in a subject in need thereof, comprising administering a therapeutically effective amount of a biocompatible bulking agent composition to the anal region of the subject.

In some embodiments, the composition can be administered to the anal canal wall. In some embodiments, the composition is administered to the submucosal layer of the anal canal wall. In some embodiments, the composition is administered to the deep submucosal layer in the proximal part of the high pressure zone of the anal canal about 5 mm above the dentate line. In some embodiments, the composition is administered to the deep submucosal layer in the proximal part of the high pressure zone of the anal canal at least about 5 mm above the dentate line.

In some embodiments, the composition can be administered intersphincterically to the subject. In some embodiments, the composition is administered to the anal sphincter of the subject. In some embodiments, the composition is administered to the region around the anal sphincter of the subject.

In some embodiments, the composition is administered to the subject with the aid of ultrasonic guidance.

In certain embodiments, the biocompatible bulking agent composition includes sodium hyaluronate.

In some embodiments, the biocompatible bulking agent composition includes dextranomer.

In some embodiments, the biocompatible bulking agent composition includes sodium hyaluronate and dextranomer.

In some embodiments, the biocompatible bulking agent composition contains (i) a pseudoplastic polymer carrier 0.05-50% (w/w) of the total composition; and (ii) a water insoluble, biocompatible, and biodegradable tissue augmenting substance comprising a dextranomer. In certain embodiments, the pseudoplastic polymer carrier can be selected from the group consisting of glycosaminoglycans, hydroxyl ethyl cellulose, carboxy methyl cellulose, xanthan gum, and alginates. In an exemplary embodiment, the glycosaminoglycan is hyaluronic acid.

In some embodiments, the dextranomer can be present in the form of microbeads.

In some embodiments, the biocompatible bulking agent composition further includes a therapeutically active ingredient. The therapeutically active ingredient can be in a sustained release form.

In any of the foregoing embodiments, the biocompatible bulking agent composition can be administered by injection. Injection, in various embodiments, can be through a syringe direct into a target tissue, or via a catheter or other delivery device.

In an exemplary embodiment, the biocompatible bulking agent composition can be administered by injection to the submucosal layer of the anal canal wall.

In some embodiments, the biocompatible bulking agent composition is administered by injection to the submucosal layer of the anal canal wall at a first location, and optionally at additional locations that may include a second, third, fourth, fifth, sixth, seventh, eighth, ninth, or tenth, or more locations. In some embodiments, the biocompatible bulking agent composition is administered at 2 locations, 3 locations, 4 locations, 5 locations, 6 locations, 7 location, 8 locations, 9 locations, or 10 or more locations. In some embodiments, the biocompatible bulking agent composition is administered at 4 or about 4 locations. In certain embodiments the biocompatible bulking agent is administered 1 mm to 5 cm from dentate line. In other embodiments the biocompatible bulking agent is administered from between about 5 mm to about 2 cm from the dentate line. In certain embodiments the biocompatible bulking agent is administered from between about 2 mm to about 20 mm from the dentate line and in 4 or more locations distributed about a circumference of the anal canal. In some embodiments the 4 or more locations are distributed evenly spaced from one another, in other embodiments the locations are grouped together or not evenly spaced. In some embodiments, a plurality of the 4 or more locations is distributed closer together relative to the remaining location or locations. For example, three of four locations may be closer together than a fourth location.

In some embodiments, a subject is injected with two rounds of, for example, four injections administered during each round. In one embodiment, the rounds of injections are administered at the same distance from the dentate line. In other embodiments the rounds of injections are administered at different distances from the dentate line. For example, in one embodiment, a first round of injections is administered at 5 mm from the dentate line, and a second round of injections is administered at 4 mm from the dentate line. In one embodiment, the injections from the second round are in the same location as the first round of injections. In one embodiment, the second injection sites are off-set from the locations of the first injections. In one embodiment, the injections from the second injection are in the same location as the first injections, but they are a different distance from the dentate line. In one embodiment, the second injection sites are off-set from the locations of the first injections, but they are a different distance from the dentate line. For example, the second injection sites can be administered at a distance of about 1 to 5 mm from the location of the first injection sites.

In some embodiments, the subject under treatment may be selected if the subject previously failed conservation therapy. The conservation therapy can include diet, fiber therapy, and medications for treatment of diarrhea.

The subject under treatment can be female, and in certain embodiments, preferably can be a female of non-childbearing potential. The subject can also be a female of childbearing potential but who is using a form of medically acceptable contraception and who has a negative urine pregnancy test result immediately to treated.

In some embodiments, the subject excludes those who are (a) pregnant or lactating, (b) have inflammatory bowel disease, (c) have an immunodeficiency disorder or ongoing immunosuppressive therapy, (d) have received prior radiation treatment to the pelvic area, (e) have significant mucosal or full thickness rectal prolapse, (f) have active anorectal conditions that include abscess, fissures, sepsis, bleeding, proctitis, and infection, (g) have anorectal atresia, tumors, stenosis, or malformation, (h) have rectocele, (i) have rectal varices, (j) have existing implant in anorectal region, (k) have an allergy to hyaluronic acid, (1) have anastomosis to the rectum or anus within 10 cm of the dentate line, (m) have an unstable psychological condition, or (n) have a bleeding disorder.

The subject under treatment can be male.

In some embodiments, the subject under treatment remains free from reintervention for more than 24 months, or for more than 36 months.

In some embodiments, the subject under treatment remains free from the occurrence of device-related adverse event for more than 24 months, or for more than 36 months.

In some embodiments, the subject under treatment has a favorable Fecal Incontinence Quality of Life (Rockwood Instrument) result after more than 24 months after treatment, or for more than 36 months showing said treatment is effective.

In some embodiments, the subject under treatment has a favorable Cleveland Clinic Florida Fecal Incontinence Score after more than 24 months or after more than 36 months after treatment showing said treatment is effective.

In some embodiments, the subject under treatment has a favorable Global Perceived Effect Score after more than 24 months after treatment or after more than 36 months showing said treatment is effective.

In some embodiments, the subject under treatment has a favorable Time to Fecal Incontinence Reintervention score after more than 24 months or after more than 36 months after treatment showing said treatment is effective.

In some embodiments, the biocompatible bulking agent composition shows relative anatomic stability after more than 24 months after treatment or more than 36 months after treatment showing said treatment is effective.

In some embodiments, the biocompatible bulking agent composition shows minimal local shifting. This is one indication that the treatment is effective. In one embodiment, the biocompatible bulking agent composition shows minimal local shifting after 24 months after treatment. In one embodiment, the biocompatible bulking agent composition shows minimal local shifting after 36 months after treatment. In one embodiment, the biocompatible bulking agent composition shows minimal local shifting from between 24 and 36 months after treatment.

In some embodiments, the biocompatible bulking agent composition demonstrates little or no migration to other body parts or systems. This is one indication that the treatment is effective. In one embodiment, the biocompatible bulking agent composition shows little or no migration 24 months after treatment. In one embodiment, the biocompatible bulking agent composition shows little or no migration more than 24 months after treatment.

In some embodiments, the subject under treatment show no substantial infections associated with the biocompatible bulking agent. This is one indication that the treatment is effective. In one embodiment, a subject shows no substantial infections associated with the biocompatible bulking agent after 24 months after treatment. In one embodiment, a subject shows no substantial infections associated with the biocompatible bulking agent 36 months after treatment. In one embodiment, a subject shows no substantial infections associated with the biocompatible bulking agent from between 24 and 36 months after treatment.

These and other embodiments are disclosed or are obvious from and encompassed by, the following Detailed Description.

### DETAILED DESCRIPTION

Embodiments relate to the discovery that the local administration, e.g., by injection, of a biocompatible bulking agent composition comprising dextranomer and sodium hyaluronate (e.g., Solesta®) to the wall of the anus or in the vicinity of the sphincter muscle results in safe and effective treating against incontinence, e.g., fecal incontinence, in a non-invasive manner (i.e., without requiring surgery) in a subject with incontinence for a period of time that extends beyond 24 months and even up to 36 months or more without reintervention. Prior to the invention, the safety and effectiveness of such materials beyond a period of 24 months post-administration were unknown and not predictable from known information regarding the use of such compounds for treating incontinence.

Embodiments may be understood more readily by reference to the following detailed description of and the Examples included therein. Before the present methods and techniques are disclosed and described, it is to be understood that embodiments are not limited to specific analytical or synthetic methods as such may, of course, vary. It is also to be understood that the terminology used herein is for the purpose of describing particular embodiments only and is not intended to be limiting. Unless defined otherwise, all technical and scientific terms used herein have the meaning commonly understood by one of ordinary skill in the art.

As used herein and in the appended claims, the singular forms “a,” “and,” and “the” include plural reference unless the context clearly dictates otherwise. Thus, for example, reference to “a gene” is a reference to one or more genes and includes equivalents thereof known to those skilled in the art, and so forth.

As used herein, the terms referencing “anus,” “anal canal,” or “anal region,” or the like, are intended to refer to the anatomical bodily part medically identified as the anus whose function is to control the expulsion of feces from the rectum. The opening and closing of the anus is controlled by the “anal sphincter muscles”, which keep the anus open or closed. Accordingly, “anus,” “anal canal,” or “anal region” can also encompass the anal sphincter muscles or the regions surrounding the same that control the opening and closing of the anus.

As used herein, the term “anal wall” refers to any tissue forming the structure of the anus, i.e., the tissue that is proximate the open space or channel forming the anus. The anal wall is formed of various layers of distinct tissues, including the mucosal and submucosal tissue layers.

As used herein, the term “local” or “locally,” as in local administration or local coadministration of one or more therapeutics, refers to the delivery of a therapeutic agent to a bodily site that is proximate or nearby the target or intended site of treatment. For example, local administration of a biocompatible bulking agent to the anus means that the agent is delivered directly to the anal cavity, the anal wall, including to the submucosal layer of the anal wall, or to the anal sphincters or region surrounding the same. As used herein, the term “pharmaceutically effective regimen” refers to a systematic plan for the administration of one or more therapeutic agents which includes aspects such as drug concentrations, amounts or levels, timing, and repetition, and any changes therein made during the course of the drug administration.

As used herein, the term “intracellular permeation enhancing agent” refers to a compound, molecule, or substance, or the like which increases the passage of a therapeutic agent across a cell membrane, e.g., cells of the anal wall, and thus, enables the exposure of the contents (e.g., proteins, DNA, cellular machinery) of intracellular environment to the therapeutic agent.

As used herein, the term “co-administering,” or “co-administration,” and the like refers to the act of administering two or more agents, therapeutics, compounds, therapies, or the like, at or about the same time. It may also refer to the administering or embedding of an active agent together with a biocompatible bulking agent as described herein. The order or sequence of administering the different agents, e.g., chemotherapeutics, intracellular permeation enhancing agents, or immunotherapeutic agents, may vary and is not confined to any particular sequence. Co-administering may also refer to the situation where two or more agents are administered to different regions of the body or via different delivery schemes, e.g., where a first agent is administered systemically and a second agent is administered directly to a tissue, e.g., the submucosal tissue of the anal wall.

The terms “effective amount”, “therapeutically effective amount” or “pharmaceutically effective amount” as used herein, refer to an amount of an agent or compound or composition that is sufficient to treat a disorder, e.g., fecal incontinence. In some embodiments, the result is a reduction in and/or alleviation of the signs, symptoms, or causes of a disorder, or any other desired alteration of a biological system. For example, an “effective amount” for therapeutic uses is the amount of the composition comprising a compound as disclosed herein required to provide a clinically significant decrease in a disorder.

The term “subject,” as used herein, refers to any organism that has urinary and/or fecal incontinence. Such organisms include, but are not limited to, human, dog, cat, horse, cow, sheep, goat, mouse, rat, guinea pig, monkey, avian, reptiles etc.

The term “pharmaceutically acceptable” as used herein, refers to a material, (e.g., a carrier or diluent), which does not abrogate the biological activity or properties of the compounds or compositions described herein, and is relatively nontoxic (i.e., the material is administered to an individual without causing undesirable biological effects or interacting in a deleterious manner with any of the components of the composition in which it is contained).

The term “isolated,” as used herein, refers to any composition, molecule, or mixture that has undergone a laboratory purification procedure including, but not limited to, extraction, centrifugation, chromatographic separation (i.e., for example, thin layer chromatography or high performance liquid chromatography).

As used herein, the term “regimen” refers to the various parameters that characterize how a composition is administered, including, the amount, timing, and iterations, as well as the ratio of different drugs or agents to one another.

In certain embodiments, provided herein are biocompatible bulking agents for use in treating certain conditions, including incontinence of the bowels. In some embodiments, the biocompatible bulking agents can comprise dextranomer. The biocompatible bulking agents may also comprise a pseudoplastic polymer carrier, which may include glycosaminoglycans, hydroxyl ethyl cellulose, carboxy methyl cellulose, xanthan gum, and alginates. In an exemplary embodiment, the glycosaminoglycan can be hyaluronic acid. Thus, in some embodiments, the biocompatible bulking agent can comprise both dextranomer and hyaluronic acid. In a specific embodiment, the biocompatible bulking agent is Solesta.

Any useful ratio of components of the biocompatible bulking agent as described herein is contemplated. The ratio may be referenced as the ratio of specific components to one another. The ratio may also be referenced in terms of the ratio or percentage of a single component to the total weight of the composition.

Thus, in certain embodiments, the dextranomer can be present in an amount that is equivalent to about 0.01%, 0.02%, 0.04%, 0.05%, 0.10%, 0.20%, 0.4%, 0.8%, 1.0%, or about 2%, about 4%, or about 8%, or about 16%, or about 32%, or about 50%, or about 60%, or about 70%, or about 80%, or about 90%, or about 99% or more the total weight of the composition.

The pseudoplastic polymer, likewise, can be in any suitable amount relative to the total weight of the composition, and can be, for example, at about 0.01%, 0.02%, 0.04%, 0.05%, 0.10%, 0.20%, 0.4%, 0.8%, 1.0%, or about 2%, or about 4%, or about 8%, or about 16%, or about 32%, or about 50%, or about 60%, or about 70%, or about 80%, or about 90%, or about 99% or more the total weight of the composition.

In some embodiments, the weight ratio of dextranomer to pseudoplastic polymer carrier (e.g., hyaluronic acid) can be about 0.1:1, 0.2:1, 0.3:1, 0.4:1, 0.5:1, 0.6:1, 0.7:1, 0.8:1, 0.9:1, or 1:1, or alternatively, 1:0.1, 1:0.2, 1:0.3, 1:0.4, 1:0.5, 1:0.6, 1:0.7, 1:0.8, or 1:0.9, or alternatively, 1:2, 1:5, 1:10, 1:50, 1:100, 1:200, 1:400, 1:800, or 1:1000, or more.

In certain embodiments, the biocompatible bulking agent comprises a polymer, e.g., a pseudoplastic polymer carrier, dissolved in a suitable solution, such as physiological saline, such as a matrix. The polymer can be selected from the group consisting of glycosaminoglycans, such as hyaluronic acid, hydroxy ethyl cellulose, carboxy methyl cellulose, xanthan gum, and alginates. In some embodiments, the matrix comprises about 0.05-50% (w/w) of the composition. This carrier gel can have pseudoplastic properties, i.e., it has shear thinning properties.

The tissue augmenting or bulking substance of the composition can comprise water insoluble, biodegradable and biocompatible polymers. Examples of suitable polymers include, but are not limited to, collagen, starch, dextranomer, polylactide and copolymers thereof, poly-beta-hydroxybutyrate and copolymers thereof.

The pseudoplastic properties of the carrier gel provide effective dispersion of the tissue augmenting substance therein. The dispersion can be formed at the time of injection or as a prefabricated formulation. In some embodiments, it is also desirable for the composition to comprise one or more therapeutically active ingredient(s).

Additional biocompatible bulking agents useful in the embodiments described herein can be found, for example, in U.S. Pat. No. 5,827,937, which is incorporated herein by reference in its entirety.

Embodiments are also directed to any suitable method for obtaining and/or making the biocompatible bulking agents described herein.

According to one aspect, a process for preparing a biocompatible bulking agent is provided. In some embodiments, the process provides: forming an aqueous solution of a water soluble, cross-linkable polysaccharide; initiating a cross-linking of said polysaccharide in the presence of a polyfunctional cross-linking agent therefor; sterically hindering the cross-linking reaction from being terminating before gelation occurs, an activated polysaccharide thereby being obtained; and reintroducing sterically unhindered conditions for said activated polysaccharide so as to continue the cross-linking thereof up to a viscoleastic gel.

Accordingly, this process involves a cross-linking of a water-soluble, cross-linkable polysaccharide in at least two steps or stages, where the cross-linking reaction is discontinued before the gelation is initiated, said discontinuance being accomplished by sterically hindering said cross-linking reaction. The cross-linking reaction is then continued in a second step by reintroducing sterically unhindered conditions.

It has unexpectedly been found that by said steric hindrance an activated polysaccharide is obtained, the cross-linking or polymerization of which can be continued merely by reintroducing sterically unhindered conditions therefor. Secondly, it has also unexpectedly been found that the polysaccharide gel composition obtained thereby does not form the compact, dense structure which would have been obtained if performing the corresponding cross-linking reaction in one single step to a fully cross-linked gel but rather a viscoleastic gel. Furthermore, the new gel structure obtained by the methods described herein represent a substantially irreversible gel structure which does not swell to any appreciable extent in contact with water or any other aqueous medium. Generally this means that said re-swelling is less than 10% by volume based on the volume as obtained from the described process.

Without wishing to be bound by any theory, it is believed that the new structure obtained by methods described herein is a combination of cross-linking between existing polymer chains and an elongation of existing chains rather than a very dense network giving a very rigid structure. Accordingly, a viscoelastic product is obtainable by the methods described herein.

As used herein the term “sterically hindering the cross-linking reaction” can be interpreted in a broad sense, e.g. it need not necessarily be a complete hindrance but instead can be a partial hindrance of the reaction referred to. That is, what is important is that the rate of cross-linking is substantially reduced to enable the final cross-linking reaction to take place with new reaction sites involved.

Similarly, the term “reintroducing sterically unhindered conditions” can also be interpreted broadly, generally meaning that said sterically unhindered conditions need not necessarily be exactly the same steric conditions as were used when initiating the cross-linking reaction. Thus, what is generally of importance is that said sterically unhindered conditions enable more rapid reactions to take place than said sterically hindered conditions.

The steric hindrance of the cross-linking reaction can be obtainable in different ways, but a preferred embodiment is represented by the case where the steric hindrance comprises diluting the aqueous medium in which the cross-linking reaction is performed, to accomplish a lower concentration of the polysaccharide in said medium.

The reintroduction of sterically unhindered conditions is possible in different ways, but a preferred embodiment in this respect is the case which comprises evaporating the aqueous medium in which the cross-linking reaction is performed, to accomplish a higher concentration of the polysaccharide in said medium. In some embodiments, reintroduction of sterically unhindered conditions comprises dialysing the aqueous medium in which the cross-linking reaction is performed.

In some embodiments, the steric hindrance of the cross-linking reaction is accomplished before the cross-linking agent has been consumed. This in turn generally also means that the reintroduction of sterically unhindered conditions is initiated in the presence of said non-consumed cross-linking agent.

The steric hindrance of the cross-linking reaction can generally be started or performed in the range of about 50-90% of the total gelation time used in the process according to the invention, consideration also being taken to suitable elasticity or consistency for the intended use of the composition.

Thus, the term “water-soluble” can be interpreted in a broad sense, pure water not necessarily being necessary. That is, aqueous solution means any solution wherein water is the major component (e.g. present at greater than about 50%, 60%, 70%, 80% or 90% by weight or volume). A preferred sub-group of polysaccharides in connection with the invention is, however a glycosaminoglycan, of which hyaluronic acid is an example.

The cross-linking agent to be used can be any previously known cross-linking agent useful in connection with polysaccharides, consideration being taken to ensure that the biocompatibility prerequisites are fulfilled. Preferably, however, the cross-linking agent is selected from the group consisting of aldehydes, epoxides, polyaziridyl compounds, glycidyl ethers and dividylsulfones. Of these glycidyl ethers represent an especially preferred group, of which 1,4-butandiol digylcidylether can be referred to as a preferred example. In this connection it should also be mentioned that “polyfunctional” includes difunctional.

The initial cross-linking reaction in the presence of a polyfunctional cross-linking agent can be performed at varying pH values, primarily depending on whether ether or ester reactions should be promoted. In some embodiments, the cross-linking reaction is performed at an alkaline pH, especially above about pH 9, e.g. in the range of about pH 9-12, when promoting ether formations. When promoting ester formations said cross-linking reaction is preferably performed at an acidic pH, especially at about pH 2-6.

In some embodiments, the prepared cross-linked polysaccharide gel composition is utilized so as to enable the manufacture of a viscoelastic composition. Such a viscoelastic composition is for instance useful in eye surgery, as a synovial fluid substitute, as eyedrops, etc,. Thus, by utilizing the steric technology according to the methods described herein, it is possible to obtain chain extensions, chain branchings, cross-links, etc, in a more controlled way than by previously used techniques with more or less randomized coupling sites.

Furthermore, through the fact that the gels obtained by the methods described herein do not retain their original volume in the presence of an aqueous medium, the new products do not cause any interfering or negative volume effects in these or other medical uses.

Accordingly, it is also possible to include within the polysaccharide gel composition any biologically active substance for which a polysaccharide gel carrier is desired or accepted. In this context the dilution-concentration technique used in the process claimed enables the enclosure of said biologically active substance before subjecting the polysaccharide to sterically unhindered conditions. That is, while sterically unhindered conditions generally means a concentrating operation, such an operation means that the biologically active substance is present in a phase that is more compacted than when said substance was included in said carrier. The biologically active substance can thus be retained much longer as compared to previously known gel cross-linking reactions, thereby providing a better sustained release profile for the active substance.

In connection with the incorporation of the biologically active substance into the composition an adjustment of the conditions to physiological pH and salt conditions is preferably performed to have a preparation ready for medical use. Such a physiological adjustment is preferred also as concerns the reaction conditions as the second step of the process has been found to proceed well under such conditions.

Embodiments are not limited in any respect as to the biologically active substance as compared to the use of said substance in prior cases. The condition to be treated should be decisive for the specific substance to be selected.

However, interesting substances in connection with the compositions can be selected from the group consisting of hormones, cytokines, vaccines, cells and tissue augmenting substances. Thus, the unique combination of properties of the new compositions described herein makes it extremely advantageous in connection with these substances primarily due to outstanding depot or sustained release properties and non-swelling properties.

An exemplary group of biologically active substances useful in connection with the compositions described herein include tissue augmenting substances such as, for example, a polysaccharide gel as an advantageous carrier therefore. Further details concerning such products can be found in WO94/21299, which is incorporated herein by reference. More specifically, a preferred tissue augmenting substance comprises a polymer selected from collagen, starch, dextranomer, polylactide and copolymers thereof, and poly-beta-hydroxibutyrate and copolymers thereof.

The process also enables the incorporation of the biologically active substance by chemical reaction with the polysaccharide gel structure, or the cross-linking agent therefor, provided that said active substance contains functional groups reactive therewith. Unique properties or combinations of properties can thereby be obtained as in such a case; for example, the release rate of the active ingredient can be decided by the degradation or decomposition of the polymer network rather than by the dissolution or migration rate for the substance referred to from the gel network.

A modification of last-mentioned technique means that the functional groups of the active substance may have been pre-reacted with a cross-linking agent for the polysaccharide. Preferably the same cross-linking agent is used as is used in the cross-linking of the polysaccharide.

Since the processes described herein provide a new polysaccharide gel composition or structure, another aspect is represented by the novel polysaccharidc gel composition prepared therefrom. In this respect the scope of protection encompasses not only the polysaccharide gel composition whenever prepared by said process but also any polysaccharide gel composition which is obtainable by a similar technique.

Accordingly, provided herein is a cross-linked biocompatible polysaccharide gel composition (otherwise referred to as a “biocompatible bulking agent” or “biocompatible bulking agent composition” herein), which is obtainable by cross-linking of a cross-linkable polysaccharide with a polyfunctional cross-linking agent therefor in two steps, the first cross-linking step being terminated before gelation occurs, by a steric hindrance of the cross-linking reaction, and the second cross-linking step being initiated by reintroducing sterically unhindered conditions for said cross-linking reaction to continue the same up to a viscoelastic gel.

A further description of the making of embodiments of the biocompatible bulking agents may be found in U.S. Pat. No. 5,827,937, which is incorporated herein by reference.

Embodiments are also directed to methods for treating, preventing, or alleviating disorders relating to or that cause incontinence.

In one aspect, provided herein is a method to treat fecal incontinence by administering a therapeutically effective amount of a biocompatible bulking agent as described herein.

In some embodiments, the bulking agent is administered to the anal region of a subject in need thereof. In some embodiments, the bulking agent is administered to the anal canal wall. In some embodiments, the bulking agent is administered to the submucosa of the anal canal wall. In some embodiments, the bulking agent is administered intersphincterically. For example, the bulking agent can be administered to the anal sphincter or to the region around the anal sphincter. Administration of the bulking agent, or composition comprising the same, can be carried out by a direct means, such as, for example, by direct injection using a syringe or a catheter or a similar medical device.

In some embodiments, the biocompatible bulking agent is injected in the deep submucosal layer in the proximal part of the high pressure zone of the anal canal about 5 mm above the dentate line. In some embodiments, the biocompatible bulking agent is injected in the deep submucosal layer in the proximal part of the high pressure zone of the anal canal about 7.5 mm, 10 mm, 12 mm, or 15 mm above the dentate line. In some embodiments, the biocompatible bulking agent is injected in the deep submucosal layer in the proximal part of the high pressure zone of the anal canal up to about 10 mm, 15 mm, 20 mm or 25 mm above the dentate line.

In some embodiments, administration of the biocompatible bulking agent, or a composition comprising the same, is guided with the aid of an ultrasound or ultrasonic device.

Preferably, a total of four submucosal injections of 1 mL of the biocompatible bulking agent composition are administered at each treatment session. However, the composition can be administered as a total of up to 5, 6, 7, 8, 9 or 10 injections of a specified volume of composition. In some embodiments, each injection volume is about 1 mL. In some embodiments, each injection volume can vary between about 0.25 to 2.5 mL. For example, the injection volume can be from about 0.25 to 2.5 mL, from about 0.5 to 2.0 mL, from about 0.75 to 1.5 mL, or from about 1.0 to 1.25 mL. In some embodiments, the injection volume is between about 0.5 to 2.0 mL. In some embodiments, the injection volume is between about 0.75 to 1.5 mL. In some embodiments, the injection volume is between about 0.75 to 1.25 mL. In some embodiments, the injection volume is between about 1.0 to 1.25 mL.

The injected or administered bulking agent causes a stable building or “bulking-up” of the anal wall such that the subject is capable of better controlling the release of fecal matter through the anus, thereby treating the incontinence.

The biocompatible bulking agent is stable, safe, and effective for over 24 months, and even up to 36 months and longer.

Embodiments also relate to methods for measuring or evaluating the safety and effectiveness of the compositions as described herein. Methods for evaluating the safety, effectiveness, and stability of the bulking agent (e.g., Solesta®) can include, but are not limited to, evaluating the following parameters:
Freedom from fecal incontinence reintervention measured at 6, 12, 24, and 36 months post-treatment. An effectiveness endpoint is freedom from Fecal Incontinence Reintervention. Patients can be evaluated at 6, 12, 24, and 36 months after the last treatment for the occurrence (or lack of occurrence) of Fecal Incontinence Reintervention. Fecal Incontinence Reintervention includes fecal incontinence treatment of sphincteroplasty, implantation of artificial bowel sphincter, retreatment with the bulking agent, graciloplasty, sacral nerve stimulation (SNS) or other surgical interventions that occur more than 4 months after the last primary treatment.
Occurrence of device related adverse events (AEs) measured at 6, 12, 24, and 36 months post-treatment. Another endpoint comprises the occurrence of device related AEs. Patients can be evaluated at 3, 6, 12, 24, and 36 months after the last treatment. All AEs can be collected and causal relationship to the device will be assessed by the investigator.
Fecal Incontinence Quality of Life measured at 6, 12, 24, and 36 months post-treatment. The endpoint of Fecal Incontinence Quality of Life using the Rockwood instrument can be assessed at baseline, 6, 12, and 36 months after last treatment. Patients can fill out a questionnaire at screening visit and at follow up visits. The Fecal Incontinence Quality of Life instrument is a questionnaire completed by patients that assesses the impact of quality of life as it relates to Lifestyle, Coping/Behavior, Depression/Self perception and Embarrassment. The FIQL instrument consists of a total of 29 questions.
Cleveland Clinic Florida Fecal Incontinence Score measured at 6, 12, 24, and 36 months post-treatment. The endpoint of Cleveland Clinic Florida Fecal Incontinence Score can be assessed at baseline, 6, 12, and 36 months after last treatment. The investigator can calculate the CCFIS based on a patient interview. The CCFIS is a summed score of 5 individual parameters (i.e., frequency of incontinence to gas, liquid, solid, of need to wear pad, and of lifestyle changes).
Global Perceived Effect Score measured at 6, 12, 24, and 36 months post-treatment. The endpoint of Global Perceived Effect Score can be assessed at 6, 12 and 36 months after last treatment. Global perceived effect is a subjective score and reflects the patient perception of their degree of FI after treatment compared to the period before treatment and ranges from 1-7 points.
Time to Fecal Incontinence Reintervention measured at 6, 12, 24, and 36 months post-treatment. For patients who undergo Fecal Incontinence Reintervention the endpoint of Time to Fecal Incontinence Reintervention can be measured from the date of last treatment to the date of first Fecal Incontinence Reintervention. Fecal incontinence reinterventions can include any of the following FI treatments: sphincteroplasty, implantation of artificial bowel sphincter, retreatment with the bulking agent, graciloplasty, SNS or other surgical interventions.
Anatomic stability of the bulking agent measured at 6, 12, 24, and 36 months post-treatment. Assess the relative anatomic stability of the bulking agent in a subpopulation of patients enrolled at 3-4 sites as assessed by ultrasound. Relative anatomic stability of the bulking agent can be assessed by comparing anatomical positioning of the bulking agent (or composition comprising the same), as determined by ultrasound, at the time of injection to positioning at 6 and 36 months after the last treatment to 1) confirm the presence or absence of the administered agent, and 2) determine if any local shift has occurred.
Occurrence of any peri-injection device related infectious adverse events measured up to two weeks after treatment. An endpoint can be the occurrence of peri-injection device related infectious Adverse Events for patients treated with or without prophylactic antibiotics (as decided by treating physician) prior to injection. For many applications, mucosal or submucosal delivery (e.g., to the submucosal of the anal wall) can be used for delivery of the biocompatible bulking agents as described herein, and optionally, other active agents if co-administration regimens are pursued. In certain embodiments, the compositions described herein can be formulated as solutions, emulsions or creams, ointments, gels or liposomes using the ingredients described above.

The length of treatment for a particular anal disorder will depend in part on the disorder. In one embodiment, the biocompatible bulking agent is administered in a single use format by injecting up to 4 separate doses (e.g., 1 mL solutions) into the deep submucosa of the anal wall.

The identification of those patients who are in need of treatment for an anal disorder is well within the ability and knowledge of one skilled in the art. Certain of the methods for identification of patients which are at risk of developing an anal disorder which can be treated by the subject method are appreciated in the medical arts, such as personal history, family history, travel history and expected travel plans, the presence of risk factors associated with the development of that disease state in the subject patient. A clinician skilled in the art can readily identify such candidate patients, by the use of, for example, clinical tests, physical examination and medical/family/travel history.

In certain preferred embodiments, the biocompatible bulking agents are indicated for the treatment of subjects who are 18 years of age or older and who have fecal incontinence, and in particular, who have failed conservative therapy (e.g., diet, fiber therapy, anti-motility medications). In one embodiment, subjects are selected for treatment with a biocompatible bulking agent after failing conservative therapy.

In some embodiments, the subjects who are typically treatable by the compositions and methods described herein are selected based on one or more of the following criteria:
Have fecal incontinence
Have failed conservative therapy (e.g., diet, fiber therapy, medications that treat diarrhea),
Females of non-childbearing potential; or females of childbearing potential using a form of medically acceptable contraception and have a negative urine pregnancy test at screening and immediately prior to receiving a second injection with a composition described herein.
Women who are surgically sterile or those who are post menopausal for at least 2 years prior to entering the study are not considered to be of childbearing potential. In some embodiments, contraindications for treatment with the compositions described herein can include, for example:
Active inflammatory bowel disease;
Immunodeficiency disorders or ongoing immunosuppressive therapy;
Previous radiation treatment to the pelvic area;
Significant mucosal or full thickness rectal prolapse;
Active anorectal conditions including: abscess, fissures, sepsis, bleeding, proctitis, or other infections;
Anorectal atresia, tumors, stenosis or malformation;
Rectocele;
Rectal varices;
Presence of existing implant (other than Solesta) in anorectal region; and
Allergy to hyaluronic acid based products.

In addition, in evaluating the safety and effectiveness of treatment with a composition described herein, patients who may be excluded from study in certain embodiments can include:
pregnant or lactating women;
subject with an active inflammatory bowel disease;
subject with an immunodeficiency disorder or ongoing immunosuppressive therapy;
subject who received previous radiation treatment to the pelvic area;
subject with a significant mucosal or full thickness rectal prolapse;
subject with an active anorectal conditions including: abscess, fissures, sepsis, bleeding, proctitis, or other infections;
subject with an anorectal atresia, tumors, stenosis or malformation;
subject has a rectocele;
subject has rectal varices;
subject has presence of existing implant (including Solesta®) in anorectal region;
subject has an allergy to hyaluronic acid (HA) based products;
subject has an anastomosis to the rectum or anus within 10 cm of the dentate line;
subject has an unstable condition (e.g., psychiatric disorder, a recent history of substance abuse) or otherwise thought to be unreliable or incapable of complying with the requirements of the PAS Protocol;
subject has had a sphincteroplasty within 12 months of enrollment; and
subject has any bleeding disorder.

### Pharmaceutical Preparations and Formulations

Also provided herein are compositions comprising a therapeutically effective amount of the biocompatible bulking agents as described herein.

In some embodiments, the pharmaceutical composition comprises: a biocompatible bulking agent comprising dextranomer microspheres, 50 mg/mL and stabilized sodium hyaluronate, 15 mg/mL, in a phosphate-buffered 0.9% sodium chloride solution.

In some embodiments, the biocompatible bulking agent composition is provided in a disposable syringe, e.g., a disposable 1 mL assembled glass syringe with a standard Luer-lock fitting. The syringe may be equipped with a plunger stopper, a plunger rod, and a finger grip. The syringe may be packed in a pouch and terminally sterilized by moist heat. In some embodiments, the syringe is labeled with visible depth markings to indicate the depth at which the composition is injected. The visible depth markings can be measured from the tip of the needle or the base of the needle and distinguished by a colored marking on the needle shaft that is biocompatible. In some embodiments, the needles are radiopaque. The depth markings may also be etched into the needle or a mold may be made with recessed or relief depth markings. The depth marking may be from between 2 to about 6 mm from the tip (distal end) of the needle. The depth marking may be from between 3 to about 6 mm from the tip (distal end) of the needle. The depth marking may be at about 5 mm from the tip (distal end) of the needle.

In some embodiments, provided herein is a kit or pharmaceutical product that comprises a carton or other container with a suitable number of pouches with syringes (containing a composition of biocompatible bulking agent) (e.g., 4 pouches with syringes), a suitable number of sterile needles (e.g., Sterican®, 21G×4¾ inches, 0.80×120 mm) (e.g., 5 sterile needles), patient record labels and a package insert. In certain embodiments, the kit or pharmaceutical product is for single use.

In certain embodiments, the dextranomer component of the composition consists of microspheres of dextran chains cross-linked into a three-dimensional network. The stabilized sodium hyaluronate accounts for the viscous properties of the biocompatible bulking agent and acts as a carrier that facilitates the injection of the dextranomer microspheres.

In some embodiments, the biocompatible bulking agent is injected in the deep submucosal layer in the proximal part of the high pressure zone of the anal canal about 5 mm above the dentate line. Preferably, a total of four submucosal injections of 1 mL of the biocompatible bulking agent composition are administered at each treatment session.

In some embodiments, the biocompatible bulking agent is not injected intravascularly. Injection of the biocompatible bulking into blood vessels may cause vascular occlusion. In some embodiments, the biocompatible bulking agent is not injected in the midline of the anterior wall of the rectum in men with enlarged prostate.

In some embodiments, adequate bowel preparation of the rectum using enema is suggested prior to injection. In some embodiments, adequate bowel preparation of the entire bowel, e.g., colon cleansing, is performed. The bowel cleansing may be done in split doses or the morning of the procedure only. The final doses of the bowel purgative may be dosed so that the cleansing is complete prior to the injections. One example of complete comprises the excretion of clear liquids. In addition, oral or local application antibiotic may be administered to the subject prior to the injections. Antibiotics may also be given by injection or i.v. fluids.

If a second treatment or injection is advised for a subject, the second treatment may be administered at about 2 weeks or between 2 and 3.9 weeks after the first treatment. Other embodiments and methods of administration, however, are described infra.

The following examples further demonstrate several embodiments. While the examples illustrate the invention, they are not intended to limit it.

### EXAMPLES

The structures, materials, compositions, and methods described herein are intended to be representative examples, and it will be understood that the scope of embodiments described herein is not limited by the scope of the examples. Those skilled in the art will recognize that embodiments may be practiced with variations on the disclosed structures, materials, compositions and methods, and such variations are regarded as within the ambit of the invention.

### Example 1

### Treatment of Fecal Incontinence

Evaluation the safety and effectiveness of an embodiment of the biocompatible bulking agent composition in the treatment of fecal incontinence through 36 months (3 years) is provided herein.

The methods provided herein assess the Long Term Safety and Efficacy of an injectable biocompatible bulking agent composition comprising sodium hyaluronate and dextranomer. The biocompatible bulking agent is administered in accordance with prescribing information on the package insert (which is incorporated herein by reference), which includes the injection of biocompatible bulking agent into the submucosal layer of the anal canal, typically in 4 injections of 1 ml each at about the same time per treatment. One treatment is administered per subject.

Without being bound by theory, it is believed that the biocompatible bulking agent works by building up or bulking up the tissue in the anal area and that by narrowing the opening of the anus, the muscles used to prevent feces from escaping may be able to be under the control of the subject.

The following methods are described:
Freedom from fecal incontinence reintervention. An endpoint is freedom from

Fecal Incontinence Reintervention. Patients are evaluated at 6, 12, 24, and 36 months after the last biocompatible bulking agent treatment for the occurrence (or lack of occurrence) of Fecal Incontinence Reintervention. Fecal Incontinence Reintervention includes fecal incontinence treatment of sphincteroplasty, implantation of artificial bowel sphincter, retreatment with biocompatible bulking agent, graciloplasty, sacral nerve stimulation (SNS) or other surgical interventions that occur more than 4 months after the last primary biocompatible bulking agent treatment.
Occurrence of device related adverse events (AEs). Another endpoint is the occurrence of device related AEs. Patients are evaluated at 3, 6, 12, 24, and 36 months after the last biocompatible bulking agent treatment. All AEs are collected, and causal relationship to the device is assessed by the investigator.
Fecal Incontinence Quality of Life. Another endpoint comprises Fecal Incontinence Quality of Life using the Rockwood instrument is assessed at baseline, 6, 12, and 36 months after last biocompatible bulking agent treatment. Patients fill out a questionnaire at screening visit and at follow up visits. The Fecal Incontinence Quality of Life (FIQL) instrument is a questionnaire completed by patients that assesses the impact of quality of life as it relates to Lifestyle, Coping/Behavior, Depression/Self perception and Embarrassment. The FIQL instrument consists of a total of 29 questions.
Cleveland Clinic Florida Fecal Incontinence Score. Another endpoint of Cleveland Clinic Florida Fecal Incontinence Score (CCFIS) is assessed at baseline, 6, 12, and 36 months after last biocompatible bulking agent treatment. The investigator calculates the CCFIS based on a patient interview. The CCFIS is a summed score of 5 individual parameters (i.e., frequency of incontinence to gas, liquid, solid, of need to wear pad, and of lifestyle changes).
Global Perceived Effect Score. Another endpoint of Global Perceived Effect Score is assessed at 6, 12 and 36 months after last biocompatible bulking agent treatment. Global perceived effect is a subjective score and reflects the patient perception of their degree of fecal incontinence after treatment compared to the period before treatment and ranges from 1-7 points.
Time to Fecal Incontinence Reintervention. For patients who undergo Fecal Incontinence Reintervention, the effectiveness endpoint of Time to Fecal Incontinence Reintervention is measured from the date of last treatment with biocompatible bulking agent to the date of first Fecal Incontinence Reintervention. Fecal incontinence reinterventions includes any of the following FI treatments: sphincteroplasty, implantation of artificial bowel sphincter, retreatment with biocompatible bulking agent, graciloplasty, SNS or other surgical interventions.
Anatomic stability of the biocompatible bulking agent. The inventors assess the relative anatomic stability of the biocompatible bulking agent. The biocompatible bulking agent is assessed in a subpopulation of patients enrolled at 3-4 sites by ultrasound. Relative anatomic stability of biocompatible bulking agent is assessed by comparing anatomical positioning of the biocompatible bulking agent implant, as determined by ultrasound, at the time of injection to positioning at 6 and 36 months after the last biocompatible bulking agent treatment to 1) confirm the presence or absence of biocompatible bulking agent implants and 2) determine if any local shift has occurred.
Occurrence of any peri-injection device related infectious adverse events. Another endpoint is the occurrence of peri-injection device related infectious Adverse Events for patients treated with or without prophylactic antibiotics (as decided by treating physician)

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the invention.

What is claimed is:

1. A method for treating fecal incontinence in a subject in need thereof, comprising administering a therapeutically effective amount of a biocompatible bulking agent composition to the anal region of the subject, wherein the composition is administered from about 1 mm to 5 cm from the dentate line.

2. The method of claim 1, wherein the composition is administered to one or more of the anal canal wall, intersphincterically, of the subject.

3. The method of claim 1, wherein the composition is administered to the submucosal layer of the anal canal wall.

4. The method of claim 3, wherein the composition is administered from about 1 mm to 5 cm from the dentate line.

5. The method of claim 3, wherein the composition is administered from about 5 mm to 2 cm from the dentate line.

6. The method of claim 3, wherein the composition is administered from about 2 mm to 20 mm from the dentate line and in at least four locations distributed about a circumference of the anal canal.

7. The method of claim 6, wherein the at least four locations are distributed evenly spaced from one another.

8. The method of claim 6, wherein the at least four locations are not evenly spaced from one another.

9. The method of claim 8, wherein a plurality of the at least four locations are distributed closer together relative to the remaining location or locations.

10. The method of claim 3, wherein the biocompatible bulking agent composition is administered by injection to the submucosal layer of the anal canal wall in at least 2-4 locations.

11. The method of claim 3, wherein the biocompatible bulking agent composition is administered by injection to the submucosal layer of the anal canal wall in about 4 locations.

12. The method of claim 1, wherein the composition is administered to the subject in two rounds of at least four injections per round.

13. The method of claim 12, wherein the two rounds of injections are administered to the submucosal layer of the anal canal wall at a same distance from the dentate line.

14. The method of claim 12, wherein the two rounds of injections are administered to the submucosal layer of the anal canal wall at different distances from the dentate line.

15. The method of claim 12, wherein the two rounds of injections are administered to separate locations of the anal region.

16. The method of claim 12, wherein sites of the second round of injections is administered at a distance of about 1 to 5 mm relative to the sites of the first round of injections.

17. The method of claim 1, wherein the composition is administered to the subject with the aid of ultrasonic guidance.

18. The method of claim 1, wherein the biocompatible bulking agent composition comprises sodium hyaluronate.

19. The method of claim 1, wherein the biocompatible bulking agent composition comprises dextranomer.

20. The method of claim 1, wherein the biocompatible bulking agent composition comprises sodium hyaluronate and dextranomer.

21. The method of claim 1, wherein the biocompatible bulking agent composition comprises (i) a pseudoplastic polymer carrier 0.05-50% (w/w) of the total composition; and (ii) a water insoluble, biocompatible, and biodegradable tissue augmenting substance comprising a dextranomer.

22. The method of claim 21, wherein the pseudoplastic polymer carrier is selected from the group consisting of glycosaminoglycans, hydroxyl ethyl cellulose, carboxy methyl cellulose, xanthan gum, and alginates.

23. The method of claim 22, wherein the glycosaminoglycan is hyaluronic acid.

24. The method of claim 20, wherein the dextranomer is present in the form of microbeads.

25. The method of claim 1, wherein the biocompatible bulking agent composition is administered by injection.

26. The method of claim 1, wherein the subject excludes those who are (a) pregnant or lactating, (b) have inflammatory bowel disease, (c) have an immunodeficiency disorder or ongoing immunosuppressive therapy, (d) have received prior radiation treatment to the pelvic area, (e) have significant mucosal or full thickness rectal prolapse, (f) have active anorectal conditions that include abscess, fissures, sepsis, bleeding, proctitis, and infection, (g) have anorectal atresia, tumors, stenosis, or malformation, (h) have rectocele, (i) have rectal varices, (j) have existing implant in anorectal region, (k) have an allergy of hyaluronic acid, (l) have anastomosis to the rectum or anus within 10 cm of the dentate line, (m) have an unstable psychological condition, or (n) have a bleeding disorder.

27. The method of claim 1, wherein the subject remains free from reintervention for more than 24 months.

28. The method of claim 1, wherein the subject remains free from the occurrence of device-related adverse event for more than 24 months.

29. The method of claim 1, wherein the subject has a favorable Fecal Incontinence Quality of Life (Rockwood Instrument) result after more than 24 months after treatment, has a favorable Cleveland Clinic Florida Fecal Incontinence Score after more than 24 months after treatment, has a favorable Global Perceived Effect Score after more than 24 months after treatment, has a favorable Time to Fecal Incontinence Reintervention score after more than 24 months after treatment, or wherein the biocompatible bulking agent composition shows relative anatomic stability after more than 24 months after treatment, thereby showing said treatment is effective.

30. The method of claim 1, wherein the subject shows no substantial infections associated with the biocompatible bulking agent after more than 24 months after treatment showing said treatment is effective.
